SEQUENCE LISTING (12 sequences)

SEQ ID NO: 1 - DNA, 804 bp, synthetic construct
  source 1..804: mol_type = other DNA
tgctttctctgaccagcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggc

SEQ ID NO: 2 - DNA, 508 bp, synthetic construct
  source 1..508: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 3 - DNA, 603 bp, synthetic construct
  source 1..603: mol_type = other DNA
atggctttcacagagcattcaccgctgacccctcaccgtcgggacctctgtagccgctctatctggctagcaaggaagattcgttcagacctgactgctcttacggaatcctatgtgaagcatcagggcctgaacaagaacatcaacctggactctgcggatgggatgccagtggcaagcactgatcagtggagtgagctgaccgaggcagagcgactccaagagaaccttcaagcttatcgtaccttccatgttttgttggccaggctcttagaagaccagcaggtgcattttaccccaaccgaaggtgacttccatcaagctatacatacccttcttctccaagtcgctgcctttgcataccagatagaggagttaatgatactcctggaatacaagatcccccgcaatgaggctgatgggatgcctattaatgttggagatggtggtctctttgagaagaagctgtggggcctaaaggtgctgcaggagctttcacagtggacagtaaggtccatccatgaccttcgtttcatttcttctcatcagactgggatcccagcacgtgggagccattatattgctaacaacaagaaaatgtag

SEQ ID NO: 4 - DNA, 122 bp, synthetic construct
  source 1..122: mol_type = other DNA
aacttgtttattgcagcttataatggttacaaataaagcaatagcatcacaaatttcacaaataaagcatttttttcactgcattctagttgtggtttgtccaaactcatcaatgtatctta

SEQ ID NO: 5 - DNA, 241 bp, synthetic construct
  source 1..241: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 6 - DNA, 60 bp, synthetic construct
  source 1..60: mol_type = other DNA
ccggtcgtaccgtgagtaataatgcgctcgagcgcattattactcacggtacgatttttg

SEQ ID NO: 7 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 8 - DNA, 837 bp, synthetic construct
  source 1..837: mol_type = other DNA
actagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctct

SEQ ID NO: 9 - DNA, 23 bp, synthetic construct
  source 1..23: mol_type = other DNA
gaagattcgttcagacctgactg

SEQ ID NO: 10 - DNA, 21 bp, synthetic construct
  source 1..21: mol_type = other DNA
aaggttctcttggagtcgctc

SEQ ID NO: 11 - DNA, 24 bp, synthetic construct
  source 1..24: mol_type = other DNA
tatggttgttctcgactccttcac

SEQ ID NO: 12 - DNA, 23 bp, synthetic construct
  source 1..23: mol_type = other DNA
tcgtctgtcgtaccgtgagtaat

(19) United States
(12) Patent Application Publication
(10) Pub. No.: US 2024/0252687 A1
(43) Pub. Date: Aug. 1, 2024

(54) GENE THERAPY FOR WET AGE-RELATED MACULAR DEGENERATION USING IPSC-DERIVED CELLS AS VECTORS

(71) Applicant: Allife Medicine (Zhuhai) Limited, Hengqin New District Zhuhai (CN)

(72) Inventors: Lida WU, Hengqin New District Zhuhai (CN); Yuchun Gu, Hengqin New District Zhuhai (CN)

(73) Assignee: Allife Medicine (Zhuhai) Limited, Hengqin New District Zhuhai (CN)

(21) Appl. No.: 18/433,104

(22) Filed: Feb. 5, 2024

Related U.S. Application Data
(63) Continuation of application No. PCT/CN2022/107879, filed on Jul. 26, 2022.

(30) Foreign Application Priority Data
Aug. 5, 2021 (CN) 202110894938.2

(51) Int. Cl. A61K 48/00; A61K 35/545; A61K 45/06; C07K 14/475; C07K 14/48; C12N 5/079; C12N 9/22; C12N 15/113; C12N 15/85; C12N 15/90

(52) U.S. Cl. CPC A61K 48/0066; A61K 35/545; A61K 45/06; A61K 48/0058; C07K 14/475; C07K 14/48; C12N 5/0621; C12N 9/22; C12N 15/113; C12N 15/85; C12N 15/907; C12N 2310/20; C12N 2500/32; C12N 2500/38; C12N 2501/999; C12N 2506/45; C12N 2510/00

(57) ABSTRACT

The present invention relates to gene therapy for wet age-related macular degeneration using iPSC-derived cells as vectors. CRISPR technology is used to perform site-directed dual gene editing of iPSCs, neurotrophic factors CNTF and miR-126 are expressed at the same time, and gene-edited IPSCs are then induced to differentiate into RPE cells. The RPE cells obtained by the induced differentiation can repair damaged RPE cells on CNV and inhibit the generation of choroidal neovascularization. In addition, the RPE cells can express the neurotrophic factors CNTF and miR-126 to fundamentally treat wet age-related macular degeneration, which has very good clinical application prospects.

### SEQUENCE LISTING

The instant application contains a sequence listing which has been submitted electronically in the XML file format and is herein incorporated by reference in its entirety. (Filename: “DOP2422879003PUS.20240131.Sequence Listing”; Date created: Feb. 2, 2024; File size: 14,939 Bytes).

### FIELD

The present disclosure belongs to the technical field of biomedicine, and specifically relates to gene therapy for wet age-related macular degeneration using iPSC-derived cells as vectors.

### BACKGROUND

Age-related macular degeneration (AMD), also known as senile macular degeneration, is a common, chronic, progressive blinding eye disease. According to data from the World Health Organization (WHO), AMD is estimated to be responsible for approximately 10% of global vision loss. It is predicted that AMD patients will increase to nearly 300 million people by 2040 (Wong W L, Su X, Li X, et al. Global prevalence of age-related macular degeneration and disease burden projection for 2020 and 2040: A systematic review and meta-analysis[J]. Lancet Glob Health, 2014, 2: 106-116). AMD is a multifactorial disease that primarily affects individuals over the age of 50. The disease occurs in both eyes successively or simultaneously. It mainly affects the retinal pigment epithelium, photoreceptors, Bruch's membrane and choriocapillaris. Factors such as smoking, environmental factors, family genetics, metabolism, nutritional disorders, and long-term chronic light damage to the retinal macula are also associated with AMD.

Age-related macular degeneration (AMD) is classified into two types based on clinical manifestations and fundus pathological changes: dry age-related macular degeneration (dAMD) and wet age-related macular degeneration (wAMD). Severe central vision loss is mostly caused by wAMD. wAMD, also known as exudative or neovascular macular degeneration, causes severe damage to visual function and seriously endangers the visual health of the elderly. wAMD is characterized by the formation of abnormal choroidal neovascularization (CNV) under the macula, which is located in the center of the retina. CNV can cause pathological changes such as rupture, bleeding, exudation and edema. These pathological changes damage the retinal pigment epithelium (RPE) cells on the CNV, specifically compromising the nutritional effect of RPE cells on the retinal photoreceptor cells. This results in the destruction of the retinal photoreceptor cells and damage to visual function.

The pathogenesis of wAMD is very complex. Increased intraocular levels of vascular endothelial growth factor (VEGF) are the main cause of choroidal neovascularization. As a result, the current primary treatment method for wAMD involves intraocular injection of anti-VEGF drugs. However, because intraocular injection of anti-VEGF drugs requires multiple repeated treatments and is expensive, it is difficult for patients to bear the huge financial and mental burdens. Although anti-VEGF treatment can alleviate the disease to a certain extent, it still cannot completely control the progression of the disease or fundamentally solve the vision loss caused by damage to RPE cells. Surgical removal of CNV is also one of the clinical methods to treat wAMD. The surgical method is more direct than the treatment with anti-VEGF drugs. However, surgical removal of CNV also removes the RPE cells underneath it, leading to retinal choroidal atrophy and preventing any improvement in the patient's vision after the surgery.

The present disclosure aims to apply CRISPR technology to perform site-directed dual-gene gene editing of iPSCs in the meaningless region of the 19th pair of chromosomes (AAVS region), while expressing neurotrophic factors CNTF and miR-126, and then differentiate the gene-edited iPSCs into RPE cells, which will then be transplanted to treat wet age-related macular degeneration, fundamentally addressing the vision loss caused by damage to RPE cells.

### SUMMARY

In order to solve the above-mentioned problems currently faced in this field, the present disclosure apply cells derived from iPSCs as vectors to perform gene therapy for wet age-related macular degeneration. The present disclosure provides iPSCs that have undergone site-directed dual-gene gene editing, and then differentiate them into RPE cells. The obtained RPE cells can express the neurotrophic factors CNTF and miR-126. CNTF can promote the survival of rod photoreceptors, and can also provide neurotrophic functions for retinal ganglion cells (RGCs). In addition, CNTF also helps reduce retinal edema and helps the retina reattach to the detached RPE by injecting more fluid from the retina to the choroid, thereby improving transplantation efficiency. miR-126 can inhibit the expression of VEGF in RPE cells and adjacent cells, thereby inhibiting the generation of choroidal neovascularization, which then achieves the purpose of effectively treating wet age-related macular degeneration.

The above objects of the present disclosure are achieved through the following technical solutions:

A first aspect of the present disclosure provides a construct for engineering cells to obtain gene-edited cells.

Further, the construct comprises nucleotides encoding a gene of a neurotrophic factor or an analog thereof, and/or nucleotides of anti-angiogenic nucleic acid.

Preferably, the neurotrophic factor or the analog thereof includes CNTF, NGF, BDNF, NT-3, NT-4/5, NT-6, PEDF, GDNF, GMFB, NRG1, CHRNB3, or NTR 368.

Preferably, the anti-angiogenic nucleic acid includes miR-126, miR-15/107 family, miR-17˜92 family, miR-21, miR-132, miR-296, miR-378, miR-519c, miR-210, miR-222, miR-100, miR-23, miR-27, miR-31, miR-150, miR-146a, or miR-23a.

More preferably, the neurotrophic factor or the analog thereof is CNTF.

More preferably, the anti-angiogenic nucleic acid is miR-126.

Most preferably, the construct comprises nucleotides encoding CNTF gene, and nucleotides of miR-126.

Most preferably, the nucleotides encoding CNTF gene have a sequence shown in SEQ ID NO: 3.

Most preferably, the nucleotides of miR-126 have a sequence shown in SEQ ID NO: 6.

Preferably, the construct further comprises one or more exogenous promoters, and/or one or more endogenous promoters in a selected site.

More preferably, the nucleotides encoding CNTF gene and the nucleotides of miR-126 are operably linked to one or more exogenous promoters in the construct, or to one or more endogenous promoters in a selected site.

Most preferably, the nucleotides encoding CNTF gene and the nucleotides of miR-126 are operably linked to one or more exogenous promoters in the construct.

Most preferably, the nucleotides encoding CNTF gene and the nucleotides of miR-126 are operably linked to two exogenous promoters in the construct.

Most preferably, the exogenous promoter includes CMV promoter, U6 promoter, EF1α promoter, PGK promoter, CAG promoter, UBC promoter, SV40 promoter, Human beta actin promoter, TEF1 promoter, GDS promoter, H1 promoter, U6 promoter, T7 promoter, TERT promoter, RSV promoter, or PGK1 promoter.

Most preferably, the exogenous promoter is CMV promoter or U6 promoter.

Most preferably, the CMV promoter has a nucleotide sequence shown in SEQ ID NO: 2.

Most preferably, the U6 promoter has a nucleotide sequence shown in SEQ ID NO: 5.

Preferably, the construct further comprises a pair of homology arms.

More preferably, the homology arm is specific for a selected site.

More preferably, the homology arm comprises left homology arm HA-L and right homology arm HA-R that specifically target a selected site.

Most preferably, the left homology arm HA-L has a nucleotide sequence shown in SEQ ID NO: 1.

Most preferably, the right homology arm HA-R has a nucleotide sequence shown in SEQ ID NO: 8.

Preferably, the construct further comprises one or more exogenous terminators, and/or one or more endogenous terminators in a selected site.

More preferably, the nucleotides encoding CNTF gene and the nucleotides of miR-126 are operably linked to one or more exogenous terminators in the construct, and/or one or more endogenous terminators in a selected site.

Most preferably, the nucleotides encoding CNTF gene and the nucleotides of miR-126 are operably linked to one or more exogenous terminators in the construct.

Most preferably, the nucleotides encoding CNTF gene and the nucleotides of miR-126 are operably linked to two exogenous terminators in the construct.

Most preferably, the exogenous terminator includes PolyA terminator, NOS terminator, T7 transcription terminator, rmB terminator, TO terminator, SV40 terminator, hGH terminator, BGH terminator, or rbGlob terminator.

Most preferably, the exogenous terminator is a PolyA terminator.

Most preferably, the PolyA terminator comprises PolyA element 1 and PolyA element 2.

Most preferably, the PolyA element 1 has a nucleotide sequence shown in SEQ ID NO: 4.

Most preferably, the PolyA element 2 has a nucleotide sequence shown in SEQ ID NO: 7.

Most preferably, the selected site is a safe harbor locus.

Most preferably, the safe harbor locus includes AAVS1, CCR5, ROSA26, HTRP, H11, TCR, RUNX1, β-2 microglobulin, collagen, GAPDH, or loci that meet genomic safe harbor criteria.

Most preferably, the safe harbor locus is AAVS1.

Most preferably, the construct comprises the left homology arm HA-L, the CMV promoter, the nucleotides encoding CNTF gene, the PolyA element 1, the U6 promoter, the nucleotides of miR-126, the PolyA element 2, and the right homology arm HA-R connected in series.

A second aspect of the present disclosure provides an expression vector or cloning vector.

Further, the expression vector comprises the construct in the first aspect of the present disclosure, and the cloning vector comprises nucleotides encoding a gene of a neurotrophic factor or an analog thereof, and/or nucleotides of anti-angiogenic nucleic acid.

Preferably, the neurotrophic factor or the analog thereof includes CNTF, NGF, BDNF, NT-3, NT-4/5, NT-6, PEDF, GDNF, GMFB, NRG1, CHRNB3, or NTR 368.

Preferably, the anti-angiogenic nucleic acid includes miR-126, miR-15/107 family, miR-17˜92 family, miR-21, miR-132, miR-296, miR-378, miR-519c, miR-210, miR-222, miR-100, miR-23, miR-27, miR-31, miR-150, miR-146a, or miR-23a.

More preferably, the neurotrophic factor or the analog thereof is CNTF.

More preferably, the anti-angiogenic nucleic acid is miR-126.

Most preferably, the cloning vector comprises nucleotides encoding CNTF gene and nucleotides of miR-126.

Most preferably, the nucleotides encoding CNTF gene have a sequence shown in SEQ ID NO: 3.

Most preferably, the nucleotides of miR-126 have a sequence shown in SEQ ID NO: 6.

Preferably, the expression vector further includes a vector.

More preferably, the vector includes donor vector, a DNA vector, or a viral vector.

Most preferably, the DNA vector includes a DNA plasmid carrier, a liposome binding to a DNA plasmid, a molecular conjugate binding to a DNA plasmid, or a polymer binding to a DNA plasmid.

Most preferably, the viral vectors include adenovirus vector, adeno-associated virus vector, lentiviral vector, retroviral vector, herpes simplex virus vector, baculovirus vector, Sendai virus vector, poxvirus vector, or geminivirus vector.

Preferably, the cloning vector further comprises a vector.

More preferably, the vector includes a DNA plasmid vector, a phage vector, a yeast artificial chromosome vector, or a phage-plasmid hybrid vector.

A third aspect of the present disclosure provides an engineered host cell or a population thereof.

Further, the engineered host cell or the population thereof expresses CNTF and/or miR-126.

Preferably, the engineered host cell or the population thereof overexpresses CNTF and/or miR-126.

More preferably, the engineered host cell or the population thereof overexpresses CNTF and miR-126.

Preferably, the engineered host cell or the population thereof comprises the construct in the first aspect of the present disclosure, and/or the expression vector or cloning vector in the second aspect of the present disclosure.

Preferably, the host cell includes iPSCs, embryonic stem cells, mesenchymal stem cells, adult stem cells, or tissue-specific stem cells.

More preferably, the host cell is iPSCs.

A fourth aspect of the present disclosure provides a terminally differentiated cell or a precursor cell thereof, or a population thereof.

Further, the terminally differentiated cell or the precursor cell thereof or the population thereof expresses CNTF and/or miR-126.

Preferably, the terminally differentiated cell or the precursor cell thereof or the population thereof overexpresses CNTF and/or miR-126.

More preferably, the terminally differentiated cell or the precursor cell thereof or the population thereof overexpresses CNTF and miR-126.

Preferably, the terminally differentiated cell or the precursor cell thereof or the population thereof is obtained by inducing differentiation of the engineered host cell or the population thereof in the third aspect of the present disclosure.

Preferably, the terminally differentiated cell or the precursor cell thereof includes retinal pigment epithelial cells, cone cells, rod cells, mesenchymal stem cells, photoreceptor progenitor cells, corneal epithelial cells, choroidal endothelial cells, retinal cells, corneal cells, lens cells, ganglion cells, optic nerve cells, or choroidal cells.

More preferably, the terminally differentiated cell or the precursor cell thereof is retinal pigment epithelial cells.

Furthermore, the “iPSC” used in the present disclosure refers to induced pluripotent stem cells, which are a kind of stem cells that are obtained by reprogramming somatic cells and have the ability of self-renewal and the potential to differentiate into cells of the three germ layers. Reprogramming refers to the process of obtaining induced pluripotent stem cells through exogenous gene expression, compound induction, epigenetic modification, etc.

Furthermore, the “terminal differentiated cells” used in the present disclosure refer to fully differentiated cells obtained after differentiation of iPSCs, embryonic stem cells, mesenchymal stem cells, adult stem cells, tissue-specific stem cells or cell populations thereof. In specific embodiments of the present disclosure, the stem cells are iPSCs, and the terminally differentiated cells include retinal pigment epithelial cells, cone cells, rod cells, photoreceptor progenitor cells, corneal epithelial cells, choroidal endothelial cells, retinal cells, corneal cells, lens cells, ganglion cells, optic nerve cells, or choroidal cells, preferably retinal pigment epithelial cells.

Furthermore, the “precursor cells” used in the present disclosure refer to all intermediate cells in the process of inducing differentiation of iPSCs, embryonic stem cells, mesenchymal stem cells, adult stem cells, or tissue-specific stem cells to produce terminally differentiated cells. The intermediate cells include all cells involved in the process of inducing differentiation except initial cells (iPSCs, embryonic stem cells, mesenchymal stem cells, adult stem cells, tissue-specific stem cells) and terminal cells (terminal differentiated cells).

A fifth aspect of the present disclosure provides a method for producing the engineered host cell or the population thereof in the third aspect of the present disclosure.

Further, the method comprises delivering the expression vector in the second aspect of the present disclosure and CRISPR/Cas vector targeting a selected site into a host cell.

Preferably, the selected site is a safe harbor locus.

More preferably, the safe harbor locus includes AAVS1, CCR5, ROSA26, HTRP, H11, TCR, RUNX1, β-2 microglobulin, collagen, GAPDH, and loci that meet genomic safe harbor criteria.

Most preferably, the safe harbor locus is AAVS1.

Most preferably, the CRISPR/Cas vector targeting the selected site is a CRISPR/Cas vector targeting AAVS1.

Most preferably, the CRISPR/Cas vector targeting AAVS1 is AAVS1 T2 CRIPR in pX330 vector.

Preferably, the delivery is achieved by introducing the expression vector in the second aspect of the present disclosure into a host cell.

More preferably, the introduction is performed by a method selected from the group consisting of electrotransfection method, microinjection method, ultrasound-mediated method, sonoporation method, photoporation method, magnetic transfer method, heat shock method, calcium phosphate method, liposome and polymer method, nanoparticle method, and virus transformation method.

Most preferably, the introduction is performed by electrotransfection method.

Most preferably, an electrotransfection system used in the electrotransfection method comprises, for 100 μL electroporation system, 1×10⁶ host cells, 1 g of AAVS1 T2 CRIPR in pX330 vector, and 1 μg of the expression vector in the second aspect of the present disclosure. Most preferably, the voltage used in the electrotransfection method is 1200 V.

Preferably, the method further comprises culturing and screening the host cell after delivering the expression vector in the second aspect of the present disclosure and the CRISPR/Cas vector targeting the selected site.

More preferably, the culturing is performed using E8 medium as a culture medium, and the culture medium is renewed every day.

More preferably, the culturing is performed at 37° C. and 5% CO₂.

More preferably, the culture is performed for 48-72 h.

More preferably, the screening is performed using puromycin.

More preferably, the screening is performed for 5-7 days.

Preferably, the host cell includes iPSCs, embryonic stem cells, mesenchymal stem cells, adult stem cells, or tissue-specific stem cells.

More preferably, the host cell is iPSCs.

A sixth aspect of the present disclosure provides a method for producing the terminally differentiated cell or the precursor cell thereof or the population thereof in the fourth aspect of the present disclosure.

Further, the method comprises inducing differentiation of the engineered host cell or the population thereof in the third aspect of the present disclosure.

Preferably, inducing differentiation comprises steps of:

(1) On day 0, culturing the engineered host cell or the population thereof in the third aspect of the present disclosure in RDM1 culture medium supplemented with small molecule additives.

(2) On days 1-6, renewing RDM1 culture medium every day.

(3) On days 7-12, replacing the culture medium with RDM2 culture medium supplemented with small molecule additives, and renewing RDM2 culture medium every day.

(4) On days 13-17, replacing the culture medium with RDM3 culture medium supplemented with small molecule additives, and renewing RDM3 culture medium every day.

(5) On days 18-24, replacing the culture medium with RDM4 culture medium, and renewing RDM4 culture medium every day.

(6) On days 25-36, replacing the culture medium with RMM culture medium, and renewing RMM culture medium every day.

(7) After day 37, discarding the culture medium, adding Trypsin-EDTA, and culturing.

(8) Discarding Trypsin-EDTA and adding REM culture medium to terminate digestion.

Preferably, the engineered host cell or the population thereof in the third aspect of the present disclosure in step (1) is at a cell density of 1.0×10³-5.0×10⁵/cm².

More preferably, the engineered host cell or the population thereof in the third aspect of the present disclosure in step (1) is at a cell density of 5.0×10³/cm².

Preferably, the culturing in step (1) is performed at 37° C. and 5% CO₂.

Preferably, the RDM1 culture medium in step (1) comprises 88% DMEM/F12, 10% KSR, 5 mM Monothioglycerol Solution, 1% Chemically Defned Lipid Concentrate, and 1% L-glutamine.

Preferably, the small molecule additives in step (1) are noggin, XAV-939, and LY2109761.

Preferably, the RDM2 culture medium in step (3) comprises 88% DMEM/F12, 10% KSR, 5 mM Monothioglycerol Solution, 1% Chemically Defned Lipid Concentrate, and 1% L-glutamine.

Preferably, the small molecule additives in step (3) are 6-bromoindirubin-3′-oxime (BIO), SU5402, and Thiazovivin.

Preferably, the RDM3 culture medium in step (4) comprises 88% DMEM/F12, 10% KSR, 5 mM Monothioglycerol Solution, 1% Chemically Defned Lipid Concentrate, and 1% L-glutamine.

Preferably, the small molecule additives in step (4) are 6-bromoindirubin-3′-oxime (BIO), SU5402, Thiazovivin, and Vitamin B3.

Preferably, the RDM4 culture medium in step (5) comprises 89% DMEM/F12, 10% KSR, 1% N2 medium, 1% L-glutamine, and 10 mM Vitamin B3.

Preferably, the RMM culture medium in step (6) comprises 97% DMEM/F12, 2% B27 medium, and 1% L-glutamine.

Preferably, the Trypsin-EDTA in step (7) is added at an amount of 1 mL.

Preferably, the culturing in step (7) is performed at 37° C. and 5% CO₂.

Preferably, the culturing in step (7) is performed for 10 min.

Preferably, the REM culture medium in step (8) is added at an amount of 3 mL.

Preferably, the terminally differentiated cell or the precursor cell thereof includes retinal pigment epithelial cells, cone cells, rod cells, mesenchymal stem cells, photoreceptor progenitor cells, corneal epithelial cells, choroidal endothelial cells, retinal cells, corneal cells, lens cells, ganglion cells, optic nerve cells, or choroidal cells.

More preferably, the terminally differentiated cell or the precursor cell thereof is retinal pigment epithelial cells.

A seventh aspect of the present disclosure provides a composition.

Further, the composition comprises the construct in the first aspect of the present disclosure, and/or the expression vector or cloning vector in the second aspect of the present disclosure, and/or the engineered host cell or the population thereof in the third aspect of the present disclosure, and/or the terminally differentiated cell or the precursor cell thereof or the population thereof in the fourth aspect of the present disclosure.

An eighth aspect of the present disclosure provides a pharmaceutical composition for treating and/or preventing wet age-related macular degeneration.

Further, the pharmaceutical composition comprises the expression vector or cloning vector in the second aspect of the present disclosure, and/or the engineered host cell or the population thereof in the third aspect of the present disclosure, and/or the terminally differentiated cell or the precursor cell thereof or the population thereof in the fourth aspect of the present disclosure.

Preferably, the pharmaceutical composition further comprises a pharmaceutically acceptable carrier and/or excipient.

Preferably, the pharmaceutical composition further comprises one or more therapeutic agents.

More preferably, the therapeutic agent includes peptides, cytokines, checkpoint inhibitors, mitogens, growth factors, miRNA, dsRNA, mononuclear blood cells, feeder cells, components of feeder cell or replacement factors thereof, antibodies, chemical therapeutic agents, or immunomodulatory agents.

A ninth aspect of the present disclosure provides a kit for producing an engineered host cell or a population thereof, or a terminally differentiated cell or a precursor cell thereof or a population thereof.

Further, the kit comprises the construct in the first aspect of the present disclosure, the expression vector or cloning vector in the second aspect of the present disclosure, CRISPR/Cas vector targeting AAVS1, a host cell, and one or more culture media

Preferably, the CRISPR/Cas vector targeting AAVS1 is AAVS1 T2 CRIPR in pX330 vector.

Preferably, the host cell includes iPSCs, embryonic stem cells, mesenchymal stem cells, adult stem cells, or tissue-specific stem cells.

More preferably, the host cell is iPSCs.

Preferably, the culture medium includes E8 medium, RDM1 culture medium, RDM2 culture medium, RDM3 culture medium, RDM4 culture medium, RMM culture medium, or REM culture medium.

A tenth aspect of the present disclosure provides application, selected from the group consisting of:
(1) Application of the construct in the first aspect of the present disclosure in the manufacture of an expression vector or cloning vector;
(2) Application of the construct in the first aspect of the present disclosure in the manufacture of a kit for producing an engineered host cell or a population thereof, or a terminally differentiated cell or a precursor cell thereof or a population thereof;
(3) Application of the expression vector or cloning vector in the second aspect of the present disclosure in the manufacture of an engineered host cell or a population thereof;
(4) Application of the expression vector or cloning vector in the second aspect of the present disclosure in the manufacture of a medicament for the treatment and/or prevention of wet age-related macular degeneration;
(5) Application of the expression vector or cloning vector in the second aspect of the present disclosure in the manufacture a kit for producing an engineered host cell or a population thereof, or a terminally differentiated cell or a precursor cell thereof or a population thereof;
(6) Application of the engineered host cell or the population thereof in the third aspect of the present disclosure in the manufacture of a terminally differentiated cell or a precursor cell thereof or a population thereof;
(7) Application of the engineered host cell or the population thereof in the third aspect of the present disclosure in the manufacture of a medicament for the treatment and/or prevention of wet age-related macular degeneration;
(8) Application of the terminally differentiated cell or the precursor cell thereof in the fourth aspect of the present disclosure in the manufacture of a medicament for the treatment and/or prevention of wet age-related macular degeneration;
(9) Application of the composition in the seventh aspect of the present disclosure in the manufacture of a medicament for the treatment and/or prevention of wet age-related macular degeneration;
(10) Application of the pharmaceutical composition in the eighth aspect of the present disclosure in the manufacture of a medicament for the treatment and/or prevention of wet age-related macular degeneration;
(11) Application of the kit in the ninth aspect of the present disclosure in the production of an engineered host cell or a population thereof, or a terminally differentiated cell or a precursor cell thereof or a population thereof; and
(12) Application of CNTF and/or miR-126 in the manufacture of a medicament for the treatment of wet age-related macular degeneration.

Preferably, the medicament overexpresses CNTF and/or miR-126.

More preferably, the medicament overexpresses CNTF and miR-126.

Preferably, the medicament comprises an agent that overexpresses CNTF and/or miR-126 expression.

Preferably, the medicament comprises an agent that overexpresses CNTF and miR-126 expression.

The present disclosure also provides a method for treating and/or preventing wet age-related macular degeneration.

Further, the method comprises administering to a subject in need thereof an effective amount of the expression vector or cloning vector in the second aspect of the present disclosure, and/or the engineered host cell or the population thereof in the third aspect of the present disclosure, and/or the terminally differentiated cell or the precursor cell thereof or the population thereof in the fourth aspect of the present disclosure, and/or the composition in the seventh aspect of the present disclosure, and/or the pharmaceutical composition in the eighth aspect of the present disclosure.

The present disclosure also provides a method for expressing CNTF and miR-126 in a subject in need thereof.

Further, the method comprises delivering to the subretina of a subject in need thereof an effective amount of the expression vector or cloning vector in the second aspect of the present disclosure, and/or the engineered host cell or the population thereof in the third aspect of the present disclosure, and/or the terminally differentiated cell or the precursor cell thereof or the population thereof in the fourth aspect of the present disclosure.

Compared with the prior art, the advantages and beneficial effects of the present disclosure are as follows:

(1) The RPE obtained by inducing differentiation of iPSCs prepared by the present disclosure through site-directed dual-gene gene editing can repair retinal pigment epithelial cells on CNV that have been damaged in patients with wet age-related macular degeneration after cell transplantation.

(2) After gene-edited iPSCs differentiate into RPE, they can express the neurotrophic factor CNTF. CNTF can promote the survival of rod photoreceptors, and can also provide neurotrophic functions for retinal ganglion cells (RGCs). In addition, CNTF also helps reduce retinal edema and helps the retina reattach to the detached RPE by injecting more fluid from the retina to the choroid, thereby improving transplantation efficiency.

(3) After gene-edited iPSCs differentiate into RPE, they can express miR-126, which can inhibit the expression of VEGF in RPE cells and adjacent cells, thereby inhibiting the generation of choroidal neovascularization.

Based on the above three points, the RPE obtained by differentiation of iPSCs prepared by the present disclosure through site-directed dual-gene gene editing can fundamentally treat wet age-related macular degeneration and solve the problems currently faced in this field, such as expensive intraocular injections of anti-VEGF drugs, requiring multiple repeated injections, incapable of completely controlling disease progression, and retinal choroidal atrophy caused by surgical removal of CNV which also removes the RPE cells underneath it. Thus, the present disclosure has very good clinical application prospects.

### BRIEF DESCRIPTION OF DRAWINGS

Below, the embodiments of the present disclosure are described in detail in conjunction with the accompanying drawings, wherein:

FIG. 1 shows a schematic diagram of the core structure of the HA-CNTF-U6-MIR126-HA vector constructed in the present disclosure.

FIG. 2 shows the detection results of stemness genes of iPSCs.

FIG. 3 shows the results of iPSC differentiation into RPE to form melanin-precipitated cells.

FIG. 4 shows the results of the relative expression level of CNTF mRNA in RPE cells.

FIG. 5 shows the results of the relative expression level of miR-126 in RPE cells.

FIG. 6 shows the results of the effect of RPE supernatant on tube formation of HUVEC cells. The a of FIG. 6: The result of tube formation of HUVEC cells in the control group. The b of FIG. 6: The result of tube formation of HUVEC cells after adding RPE supernatant.

### DETAILED DESCRIPTION

The present disclosure will be further described below in conjunction with specific examples, which are only used to illustrate the present disclosure and cannot be understood as limiting the present disclosure. Those of ordinary skill in the art can understand that various changes, modifications, substitutions and variants can be made to these embodiments without departing from the principles and purposes of the present disclosure. The scope of the present disclosure is defined by the claims and their equivalents. Experimental methods without specifying specific conditions in the following examples are usually tested according to conventional conditions or according to the conditions recommended by the manufacturer.

### Example 1 Construction of HA-CNTF-U6-MIR126-HA Vector

### 1. Experimental Materials

The reagent information used in the examples of the present disclosure is shown in Table 1.

TABLE 1
Reagent information used in the examples of the present disclosure

| Name | Company | Item No. |
|---|---|---|
| DMEM/F-12 with HEPES | GIBCO | 11330032 |
| Matrigel | Corning | 354277 |
| KnockOut Serum Replacement (KSR) | GIBCO | A3181502 |
| L-Glutamine (100×) | GIBCO | 35050061 |
| LDN193189 | sigma | SML0559 |
| LY2109761 | abmole | M2081 |
| XAV-939 | abmole | M1796 |
| Thiazovivin | abmole | M1856 |
| 6-bromoindirubin-3′-oxime (BIO) | abmole | M7627 |
| SU 5402 | biogems | 2159233 |
| Vitamin B3 | sigma | N3376-100G |
| N2 supplement | GIBCO | 17502001 |
| B-27 ™ Supplement, XenoFree, | Life technologies | A3353501 |
| minus vitamin A | | |
| 0.25% Trypsin-EDTA (1X) | Thermo | 25200072 |

### 2. Experimental Methods

(1) As shown in FIG. 1, the donor vector was constructed.

(2) Into the dornor vector (pShuttle (addgene, Plasmid #16402)), AAVS1 left homology arm HA-L (as shown in SEQ ID NO: 1), CMV promoter (as shown in SEQ ID NO: 2), CNTF gene sequence (as shown in SEQ ID NO: 3), polyA element 1 (as shown in SEQ ID NO: 4), U6 promoter (as shown in SEQ ID NO: 5), miR-126 framework (as shown in SEQ ID NO: 6), polyA element 2 (shown in SEQ ID NO: 7), AAVS1 right homology arm HA-R (shown in SEQ ID NO: 8) were inserted, and the constructed dornor vector was named HA-CNTF-U6-MIR126-HA.

Wherein, AAVS is a meaningless region of human chromosome 19. It does not affect the stemness and differentiation of stem cells and can be subjected to gene editing.

### Example 2 Gene Editing of iPSCs

### 1. Experimental Materials

The iPSC cells used in this example were from Beijing Chengnuo Regenerative Medicine Technology Co., Ltd.

### 2. Experimental Methods

(1) The dornor vector constructed in Example 1 and the AAVS1 T2 CRIPR in pX330 vector (addgene, Plasmid #72833) were introduced into iPSCs by electroporation.

(2) Electroporation method: 100 μL electroporation system including 1×10⁶ iPSCs, 1 g of AAVS1 T2 CRIPR in pX330 vector, and 1 μg of the dornor vector, at a voltage of 1200 V.

(3) After electroporation, E8 medium (Thermo Fisher Scientific Co., Ltd., A1517001) was used to culture the cells, and the medium was renewed every day. The culture was performed at 37° ° C. and 5% CO₂.

(4) After 48-72 h of culture, puromycin was used to screen positive cells.

(5) After 5 to 7 days of resistance screening, when the cells grew stably and no longer underwent apoptosis, single clones were selected depending on the size of the clones and placed in a 96-well plate for culture.

(6) Depending on the growth status of the cells, the single clones were gradually transferred from 96-well plates to 48-well plates, 24-well plates, 12-well plates, and 6-well plates for culture, amplification, and cryopreservation.

### Example 3 Directionally Induced Differentiation of iPSCs into RPE Cells

### 1. Experimental Materials

iPSC cells constructed by gene editing technology in Example 2 of the present disclosure.

### 2. Experimental Methods

(1) On day 0, iPSCs were plated at a density of 1.0×10³-5.0×10⁵/cm². In the present disclosure, the cells were plated at a density of 5.0×10³/cm² and then cultured in a 37° C., 5% CO₂ cell culture incubator with RDM1 as the culture medium. The basal culture medium of RDM1 comprised 88% DMEM/F12, 10% KSR, 5 mM Monothioglycerol Solution, 1% Chemically Defined Lipid Concentrate, and 1% L-glutamine, and the small molecule additives were noggin, XAV-939, and LY2109761.

(2) From day 1 to day 6, the RDM1 culture medium was renewed every day.

(3) From day 7 to day 12, RDM2 culture medium was used and renewed every day. The basal culture medium of RDM2 comprised 88% DMEM/F12, 10% KSR, 5 mM Monothioglycerol Solution, 1% Chemically Defined Lipid Concentrate, and 1% L-glutamine, and small molecule additives were 6-bromoindirubin-3′-oxime (BIO), SU5402 and Thiazovivin.

(4) From day 13 to day 17, RDM3 culture medium was used and renewed every day. The basal culture medium of RDM3 comprised 88% DMEM/F12, 10% KSR, 5 mM Monothioglycerol Solution, 1% Chemically Defined Lipid Concentrate, and 1% L-glutamine, and small molecule additives were 6-bromoindirubin-3′-oxime (BIO), SU5402, Thiazovivin, and Vitamin B3.

(5) From day 18 to day 24, RDM4 culture medium was used and renewed every day. The basal culture medium of RDM4 comprised 89% DMEM/F12, 10% KSR, 1% N2 medium, 1% L-glutamine and 10 mM Vitamin B3.

(6) From day 25 to day 36, RMM culture medium was used and renewed every day. The basal culture medium of RMM comprised 97% DMEM/F12, 2% B27 medium, and 1% L-glutamine.

(7) After day 37, the old culture medium was removed. The cells were washed twice with room temperature DPBS, then added with 1 mL of 0.25% Trypsin-EDTA preheated at 37° C., and placed in a 37° C., 5% CO₂ cell culture incubator for 10 min. Gaps were observed between individual cells under a microscope.

(8) Trypsin-EDTA was discarded, and 3 ml of REM culture medium was added to terminate digestion.

(9) The culture solution was filtered with a 35 μM filter, transferred to a 15 ml centrifuge tube and centrifuged at 1000 rpm for 5 min at room temperature.

(10) After the supernatant was discarded, the cells were gently pipetted and resuspended in REM culture medium, counted, and plated into a matrigel-coated six-well plate.

(11) From day 38 to day 51, REM was renewed every other day until the cells were collected and frozen.

### Example 4 Immunofluorescence Detection of iPSCs Constructed by Gene Editing Technology in Example 2

### 1. Experimental Materials

iPSC cells constructed by gene editing technology in Example 2 of the present disclosure.

### 2. Experimental Methods

(1) A slide with cells on it was soaked in a 24-well culture plate three times with PBS for 3 min each time.

(2) The slide was fixed with 4% paraformaldehyde at room temperature for 5 min, and soaked 3 times with PBS for 3 min each time.

(3) The slide was permeabilized with 0.5% Triton X-100 (prepared with 5% BSA) at room temperature for 20 min (this step was omitted for antigens expressed on the cell membrane).

(4) 5% BSA was used to block at room temperature for 1 hour.

(5) The blocking solution was removed. 400 μL of primary antibody was added to each well for incubation overnight at 4° C.

(6) After 12 hours, fluorescent secondary antibody was added: soaking in PBST 3 times for 5 min each time, adding diluted fluorescent secondary antibody, incubating at room temperature for 1 h, and soaking in PBST 3 times for 5 min each time.

(7) Counter-staining nuclei: DAPI was added dropwise for incubation in the dark for 5 min. The specimen was subjected to staining of nuclei, and excess DAPI was washed away with PBST 5 min×4 times.

(8) 500 μL PBS was added for the machine and film.

### 3. Experimental Results

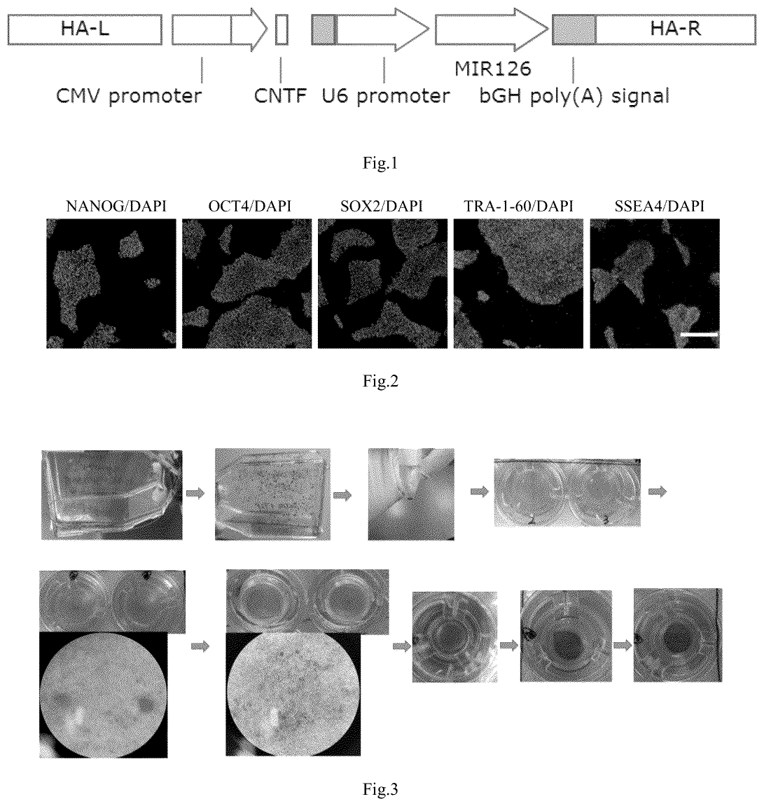

The experimental results are shown in FIG. 2. The results showed that NANOG, OCT4, SOX2, SSEA4, and TRA-1-60 were all positive, indicating that the stemness of iPSCs was not affected after gene editing.

### Example 5 qPCR Detection of Gene Expression Levels in RPE Cells Obtained by Directionally Induced Differentiation of iPSCs in Example 3

### 1. Experimental Materials

RPE cells obtained by directionally induced differentiation of iPSCs in Example 3 of the present disclosure.

### 2. Experimental Methods

(1) 200 W cells was collected and added with 1 mL TRIZOL. RNA was extracted and detected for the RNA concentration. 1 μg RNA was reverse transcribed into cDNA. Premixing was performed according to the system in Table 2.

The specific primer sequences are as follows:

CNTF gene:
The forward primer was
(SEQ ID NO: 9)
5′-GAAGATTCGTTCAGACCTGACTG-3′.
The reverse primer was
(SEQ ID NO: 10)
5′-AAGGTTCTCTTGGAGTCGCTC-3′.
miR-126 gene:
The forward primer was
(SEQ ID NO: 11)
5′-TATGGTTGTTCTCGACTCCTTCAC-3′.
The reverse primer was
(SEQ ID NO: 12)
5'-TCGTCTGTCGTACCGTGAGTAAT-3'.

TABLE 2
System composition

| Component | Volume |
|---|---|
| Forward Primer (10 μm) | 0.4 μl |
| Reverse Primer (10 μm) | 0.4 μl |
| 2×TransStar Top/Tip Green qPCR | 10 μl |
| SuperMix | |
| Nuclease-free Water | 7.2 μl |
| cDNA | 2 μl |
| Total volume | 20 μl |

(2) The above system was put into the Light cycler instrument for reaction according to the 3-step method with a number of cycles of 45. The reaction system is shown in Table 3.

TABLE 3
Reaction system

| Temperaturee | Time | Number of cycles |
|---|---|---|
| 94° C. | 30 s | |
| 94° C. | 5 s | |
| 55° C. | 15 s | 45 cycles |
| 72° C. | 10 s | |

### 3. Experimental Results

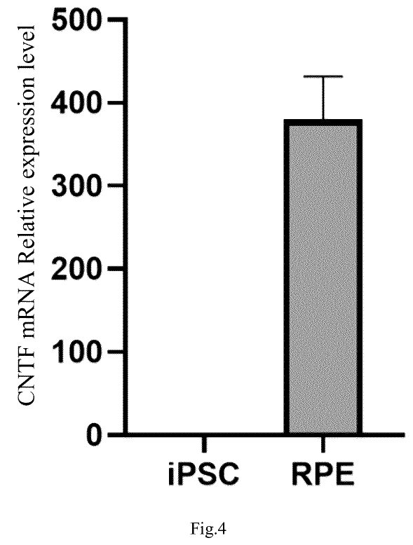

The results showed that after iPSC cells were directionally induced to differentiate into RPE cells, melanin-precipitated cells were formed (see FIG. 3). The RPE cells obtained by induced differentiation expressed a large number of genes CNTF and miR-126 (see FIGS. 4 and 5).

### Example 6 Effect of RPE Cell Supernatant Obtained from Directionally Induced Differentiation of iPSCs in Example 3 on Tube Formation of HUVEC Cells

### 1. Experimental Materials

RPE cells obtained by directionally induced differentiation of iPSCs in Example 3 of the present disclosure.

### 2. Experimental Methods

(1) An appropriate amount of HUVEC cell (promocell) suspension was used and adjusted to a cell density to 5× 10⁵/mL.

(2) 100 μL of the cell suspension per well was added to the matrigel-coated 96-well plate using 200 μL pipette tip.

(3) Half of the cells used HUVEC culture medium (promocell) (control group), and the other half of the cells used 1:1 of RPE supernatant and HUVEC culture medium (experimental group).

(4) The cell culture plate was placed at a 37° C., 5% CO₂ incubator for 24 hours of culture.

(5) After 24 h, the results of tube formation were observed.

### 3. Experimental Results

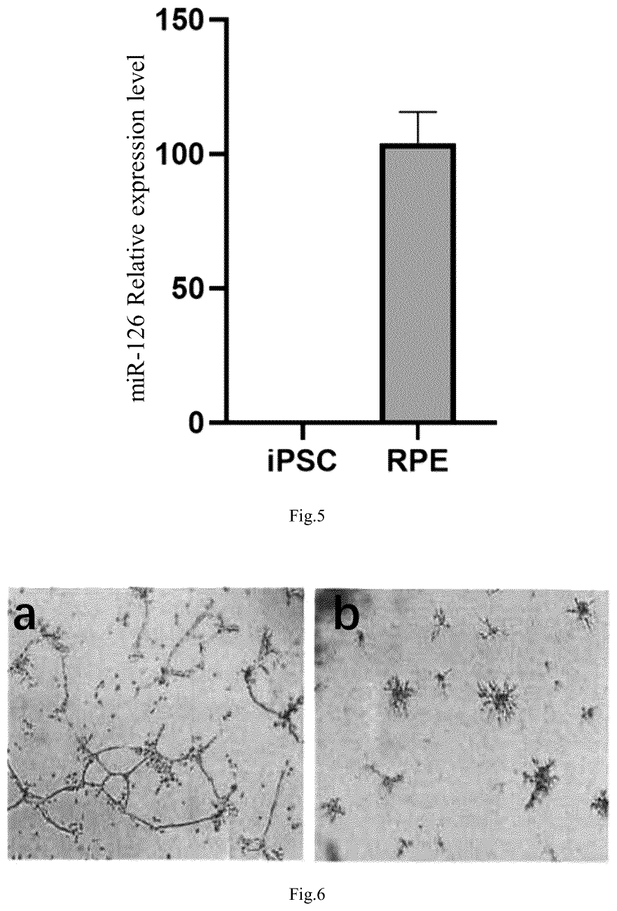

The results showed that HUVEC cells in the control group formed tubes normally (see a of FIG. 6), while the HUVEC cells added with the RPE cell supernatant obtained by directionally induced differentiation of iPSCs in Example 3 of the present disclosure could not form tubes (see b of FIG. 6), indicating that the RPE cell supernatant inhibited tube formation of endothelial cells, This further proves that the RPE cells obtained by directionally induced differentiation of iPSCs in the present disclosure have the ability to inhibit cell blood vessel formation.

The description of the above embodiments is only for understanding the method of the present disclosure and its core idea. It should be noted that those of ordinary skill in the art can make several improvements and modifications to the present disclosure without departing from the principles of the present disclosure, and these improvements and modifications will also fall within the protection scope of the claims of the present disclosure.

1. A construct for engineering cells to obtain gene-edited cells, comprising nucleotides encoding a gene of a neurotrophic factor or an analog thereof, and/or nucleotides of anti-angiogenic nucleic acid;
preferably, the neurotrophic factor or the analog thereof is selected from the group consisting of CNTF, NGF, BDNF, NT-3, NT-4/5, NT-6, PEDF, GDNF, GMFB, NRG1, CHRNB3, and NTR 368;
preferably, the anti-angiogenic nucleic acid is selected from the group consisting of miR-126, miR-15/107 family, miR-17˜92 family, miR-21, miR-132, miR-296, miR-378, miR-519c, miR-210, miR-222, miR-100, miR-23, miR-27, miR-31, miR-150, miR-146a, and miR-23a;
more preferably, the neurotrophic factor or the analog thereof is CNTF;
more preferably, the anti-angiogenic nucleic acid is miR-126;
most preferably, the construct comprises nucleotides encoding CNTF gene and nucleotides of miR-126;
most preferably, the nucleotides encoding CNTF gene have a sequence shown in SEQ ID NO: 3;
most preferably, the nucleotides of miR-126 have a sequence shown in SEQ ID NO: 6;
preferably, the construct further comprises one or more exogenous promoters, and/or one or more endogenous promoters in a selected site;
more preferably, the nucleotides encoding CNTF gene and the nucleotides of miR-126 are operably linked to one or more exogenous promoters in the construct, or to one or more endogenous promoters in a selected site;
most preferably, the nucleotides encoding CNTF gene and the nucleotides of miR-126 are operably linked to one or more exogenous promoters in the construct;
most preferably, the nucleotides encoding CNTF gene and the nucleotides of miR-126 are operably linked to two exogenous promoters in the construct;
most preferably, the exogenous promoter is selected from the group consisting of CMV promoter, U6 promoter, EF1α promoter, PGK promoter, CAG promoter, UBC promoter, SV40 promoter, Human beta actin promoter, TEF1 promoter, GDS promoter, H1 promoter, U6 promoter, T7 promoter, TERT promoter, RSV promoter, and PGK1 promoter;
most preferably, the exogenous promoter is CMV promoter or U6 promoter;
most preferably, the CMV promoter has a nucleotide sequence shown in SEQ ID NO: 2;
most preferably, the U6 promoter has a nucleotide sequence shown in SEQ ID NO: 5;
preferably, the construct further comprises a pair of homology arms;
more preferably, the homology arm is specific for a selected site;
more preferably, the homology arm comprises left homology arm HA-L and right homology arm HA-R that specifically target a selected site;
most preferably, the left homology arm HA-L has a nucleotide sequence shown in SEQ ID NO: 1;
most preferably, the right homology arm HA-R has a nucleotide sequence shown in SEQ ID NO: 8;
preferably, the construct further comprises one or more exogenous terminators, and/or one or more endogenous terminators in a selected site;
more preferably, the nucleotides encoding CNTF gene and the nucleotides of miR-126 are operably linked to one or more exogenous terminators in the construct, and/or one or more endogenous terminators in a selected site;
most preferably, the nucleotides encoding CNTF gene and the nucleotides of miR-126 are operably linked to one or more exogenous terminators in the construct;
most preferably, the nucleotides encoding CNTF gene and the nucleotides of miR-126 are operably linked to two exogenous terminators in the construct;
most preferably, the exogenous terminator is selected from the group consisting of PolyA terminator, NOS terminator, T7 transcription terminator, rmnB terminator, TO terminator, SV40 terminator, hGH terminator, BGH terminator, and rbGlob terminator;
most preferably, the exogenous terminator is PolyA terminator;
most preferably, the Poly A terminator comprises PolyA element 1 and PolyA element 2;
most preferably, the PolyA element 1 has a nucleotide sequence shown in SEQ ID NO: 4;
most preferably, the PolyA element 2 has a nucleotide sequence shown in SEQ ID NO: 7;
most preferably, the selected site is a safe harbor locus;
most preferably, the safe harbor locus is selected from the group consisting of AAVS1, CCR5, ROSA26, HTRP, H11, TCR, RUNX1, ß-2 microglobulin, collagen, GAPDH, and loci that meet genomic safe harbor criteria;
most preferably, the safe harbor locus is AAVS1;
most preferably, the construct comprises the left homology arm HA-L, the CMV promoter, the nucleotides encoding CNTF gene, the PolyA element 1, the U6 promoter, the nucleotides of miR-126, the PolyA element 2, and the right homology arm HA-R connected in series.

2. An expression vector or cloning vector, wherein the expression vector comprises the construct according to claim 1, and the cloning vector comprises nucleotides encoding a gene of a neurotrophic factor or an analog thereof, and/or nucleotides of anti-angiogenic nucleic acid;
preferably, the neurotrophic factor or the analog thereof is selected from the group consisting of CNTF, NGF, BDNF, NT-3, NT-4/5, NT-6, PEDF, GDNF, GMFB, NRG1, CHRNB3, and NTR 368;
preferably, the anti-angiogenic nucleic acid is selected from the group consisting of miR-126, miR-15/107 family, miR-17˜92 family, miR-21, miR-132, miR-296, miR-378, miR-519c, miR-210, miR-222, miR-100, miR-23, miR-27, miR-31, miR-150, miR-146a, and miR-23a;
more preferably, the neurotrophic factor or the analog thereof is CNTF;
more preferably, the anti-angiogenic nucleic acid is miR-126;
most preferably, the cloning vector comprises nucleotides encoding CNTF gene and nucleotides of miR-126;
most preferably, the nucleotides encoding CNTF gene have a sequence shown in SEQ ID NO: 3;
most preferably, the nucleotides of miR-126 have a sequence shown in SEQ ID NO: 6;
preferably, the expression vector further comprises a vector;
more preferably, the vector is selected from the group consisting of dornor vector, a DNA vector, and a viral vector;
most preferably, the DNA vector is selected from the group consisting of a DNA plasmid vector, a liposome binding to a DNA plasmid, a molecular conjugate binding to a DNA plasmid, and a polymer binding to a DNA plasmid;
most preferably, the viral vector is selected from the group consisting of adenovirus vector, adeno-associated virus vector, lentiviral vector, retroviral vector, herpes simplex virus vector, baculovirus vector, Sendai virus vector, poxvirus vector, and geminivirus vector;
preferably, the cloning vector further comprises a vector;
more preferably, the vector is selected from the group consisting of a DNA plasmid vector, a phage vector, a yeast artificial chromosome vector, and a phage-plasmid hybrid vector.

3. An engineered host cell or a population thereof, comprising the expression vector or cloning vector according to claim 2, wherein the engineered host cell or the population thereof expresses CNTF and/or miR-126;
preferably, the engineered host cell or the population thereof overexpresses CNTF and/or miR-126;
more preferably, the engineered host cell or the population thereof overexpresses CNTF and miR-126;
preferably, the host cell is selected from the group consisting of iPSCs, embryonic stem cells, mesenchymal stem cells, adult stem cells, and tissue-specific stem cells;
more preferably, the host cell is iPSCs.

4. A terminally differentiated cell or a precursor cell thereof or a population thereof, obtained by inducing differentiation of the engineered host cell or the population thereof according to claim 3, wherein the terminally differentiated cell or the precursor cell thereof or the population thereof expresses CNTF and/or miR-126;
preferably, the terminally differentiated cell or the precursor cell thereof or the population thereof overexpresses CNTF and/or miR-126;
more preferably, the terminally differentiated cell or the precursor cell thereof or the population thereof overexpresses CNTF and miR-126;
preferably, the terminally differentiated cell or the precursor cell thereof is selected from the group consisting of retinal pigment epithelial cells, cone cells, rod cells, mesenchymal stem cells, photoreceptor progenitor cells, corneal epithelial cells, choroidal endothelial cells, retinal cells, corneal cells, lens cells, ganglion cells, optic nerve cells, and choroidal cells;
more preferably, the terminally differentiated cell or the precursor cell thereof is retinal pigment epithelial cells.

5. A method for producing the engineered host cell or the population thereof according to claim 3, comprising delivering an expression vector and CRISPR/Cas vector targeting a selected site into a host cell;
preferably, the selected site is a safe harbor locus;
more preferably, the safe harbor locus is selected from the group consisting of AAVS1, CCR5, ROSA26, HTRP, H11, TCR, RUNX1, β-2 microglobulin, collagen, GAPDH, and loci that meet genomic safe harbor criteria;
most preferably, the safe harbor locus is AAVS1;
most preferably, the CRISPR/Cas vector targeting the selected site is a CRISPR/Cas vector targeting AAVS1;
most preferably, the CRISPR/Cas vector targeting AAVS1 is AAVS1 T2 CRIPR in pX330 vector;
preferably, the delivery is achieved by introducing an expression vector into a host cell;
more preferably, the introduction is performed by a method selected from the group consisting of electrotransfection method, microinjection method, ultrasound-mediated method, sonoporation method, photoporation method, magnetic transfer method, heat shock method, calcium phosphate method, liposome and polymer method, nanoparticle method, and virus transformation method;
most preferably, the introduction is performed by electrotransfection method;
most preferably, an electrotransfection system used in the electrotransfection method comprises, for 100 μL electroporation system, 1×10⁶ host cells, 1 g of AAVS1 T2 CRIPR in pX330 vector, and 1 μg of an expression vector;
most preferably, the voltage used in the electrotransfection method is 1200 V;
preferably, the method further comprises culturing and screening the host cell after delivering an expression vector and the CRISPR/Cas vector targeting the selected site;
more preferably, the culturing is performed using E8 medium as a culture medium, and the culture medium is renewed every day;
more preferably, the culturing is performed at 37° ° C. and 5% CO₂;
more preferably, the culturing is performed for 48-72 h;
more preferably, the screening is performed using puromycin;
more preferably, the screening is performed for 5-7 days;
preferably, the host cell is selected from the group consisting of iPSCs, embryonic stem cells, mesenchymal stem cells, adult stem cells, and tissue-specific stem cells;
more preferably, the host cell is iPSCs.

6. A method for producing the terminally differentiated cell or the precursor cell thereof or the population thereof according to claim 4, comprising inducing differentiation of an engineered host cell or a population thereof;
preferably, inducing differentiation comprises steps of:
(1) on day 0, culturing an engineered host cell or a population thereof in RDM1 culture medium supplemented with small molecule additives;
(2) on days 1-6, renewing RDM1 culture medium every day;
(3) on days 7-12, replacing the culture medium with RDM2 culture medium supplemented with small molecule additives, and renewing RDM2 culture medium every day;
(4) on days 13-17, replacing the culture medium with RDM3 culture medium supplemented with small molecule additives, and renewing RDM3 culture medium every day;
(5) on days 18-24, replacing the culture medium with RDM4 culture medium, and renewing RDM4 culture medium every day;
(6) on days 25-36, replacing the culture medium with RMM culture medium, and renewing RMM culture medium every day;
(7) after day 37, discarding the culture medium, adding Trypsin-EDTA, and culturing; and
(8) discarding Trypsin-EDTA and adding REM culture medium to terminate digestion;
preferably, the engineered host cell or the population thereof in step (1) is at a cell density of 1.0×10³-5.0×10⁵/cm²;
more preferably, the engineered host cell or the population thereof in step (1) is at a cell density of 5.0×10³/cm²;
preferably, the culturing in step (1) is performed at 37° C. and 5% CO₂;
preferably, the RDM1 culture medium in step (1) comprises 88% DMEM/F12, 10% KSR, 5 mM Monothioglycerol Solution, 1% Chemically Defned Lipid Concentrate, and 1% L-glutamine;
preferably, the small molecule additives in step (1) are noggin, XAV-939, and LY2109761;
preferably, the RDM2 culture medium in step (3) comprises 88% DMEM/F12, 10% KSR, 5 mM Monothioglycerol Solution, 1% Chemically Defned Lipid Concentrate, and 1% L-glutamine;
preferably, the small molecule additives in step (3) are 6-bromoindirubin-3′-oxime (BIO), SU5402, and Thiazovivin;
preferably, the RDM3 culture medium in step (4) comprises 88% DMEM/F12, 10% KSR, 5 mM Monothioglycerol Solution, 1% Chemically Defned Lipid Concentrate, and 1% L-glutamine;
preferably, the small molecule additives in step (4) are 6-bromoindirubin-3′-oxime (BIO), SU5402, Thiazovivin, and Vitamin B3;
preferably, the RDM4 culture medium in step (5) comprises 89% DMEM/F12, 10% KSR, 1% N2 medium, 1% L-glutamine, and 10 mM Vitamin B3;
preferably, the RMM culture medium in step (6) comprises 97% DMEM/F12, 2% B27 medium, and 1% L-glutamine;
preferably, the Trypsin-EDTA in step (7) is 0.25% Trypsin-EDTA preheated at 37° C.;
preferably, the Trypsin-EDTA in step (7) is added at an amount of 1 mL;
preferably, the culturing in step (7) is performed at 37° ° C. and 5% CO₂;
preferably, the culturing in step (7) is performed for 10 min;
preferably, the REM culture medium in step (8) is added at an amount of 3 mL;
preferably, the terminally differentiated cell or the precursor cell thereof is selected from the group consisting of retinal pigment epithelial cells, cone cells, rod cells, mesenchymal stem cells, photoreceptor progenitor cells, corneal epithelial cells, choroidal endothelial cells, retinal cells, corneal cells, lens cells, ganglion cells, optic nerve cells, and choroidal cells;
more preferably, the terminally differentiated cell or the precursor cell thereof is retinal pigment epithelial cells.

7. A composition, comprising a construct, and/or an expression vector or cloning vector, and/or an engineered host cell or a population thereof, and/or a terminally differentiated cell or a precursor cell thereof or a population thereof.

8. A pharmaceutical composition for treating and/or preventing wet age-related macular degeneration, comprising an expression vector or cloning vector, and/or an engineered host cell or a population thereof, and/or a terminally differentiated cell or a precursor cell thereof or a population thereof;
preferably, the pharmaceutical composition further comprises a pharmaceutically acceptable carrier and/or excipient;
preferably, the pharmaceutical composition further comprises one or more therapeutic agents;
more preferably, the therapeutic agent is selected from the group consisting of peptides, cytokines, checkpoint inhibitors, mitogens, growth factors, miRNA, dsRNA, mononuclear blood cells, feeder cells, components of feeder cell or replacement factors thereof, antibodies, chemical therapeutic agents, and immunomodulatory agents.

9. A kit for producing an engineered host cell or a population thereof, or a terminally differentiated cell or a precursor cell thereof or a population thereof, comprising a construct, an expression vector or cloning vector, CRISPR/Cas vector targeting AAVS1, a host cell, and one or more culture media;
preferably, the CRISPR/Cas vector targeting AAVS1 is AAVS1 T2 CRIPR in pX330 vector;
preferably, the host cell is selected from the group consisting of iPSCs, embryonic stem cells, mesenchymal stem cells, adult stem cells, and tissue-specific stem cells;
more preferably, the host cell is iPSCs;
preferably, the culture medium is selected from the group consisting of E8 medium, RDM1 culture medium, RDM2 culture medium, RDM3 culture medium, RDM4 culture medium, RMM culture medium, and REM culture medium.

10. Application of the construct according to claim 1, selected from the group consisting of:
(1) application of the construct in the manufacture of an expression vector or cloning vector; and
(2) application of the construct in the manufacture of a kit for producing an engineered host cell or a population thereof, or a terminally differentiated cell or a precursor cell thereof or a population thereof.

11. Application of the expression vector or cloning vector according to claim 2, selected from the group consisting of:
(1) application of the expression vector or cloning vector in the manufacture of an engineered host cell or a population thereof;
(2) application of the expression vector or cloning vector in the manufacture of a medicament for the treatment and/or prevention of wet age-related macular degeneration; and
(3) application of the expression vector or cloning vector in the manufacture a kit for producing an engineered host cell or a population thereof, or a terminally differentiated cell or a precursor cell thereof or a population thereof.

12. Application of the engineered host cell or the population thereof according to claim 3, selected from the group consisting of:
(1) application of the engineered host cell or the population thereof in the manufacture of a terminally differentiated cell or a precursor cell thereof or a population thereof; and
(2) application of the engineered host cell or the population thereof in the manufacture of a medicament for the treatment and/or prevention of wet age-related macular degeneration.

13. Application of the terminally differentiated cell or the precursor cell thereof or the population thereof according to claim 4 in the manufacture of a medicament for the treatment and/or prevention of wet age-related macular degeneration.

14. Application of the composition according to claim 7 in the manufacture of a medicament for the treatment and/or prevention of wet age-related macular degeneration.

15. Application of the pharmaceutical composition according to claim 8 in the manufacture of a medicament for the treatment and/or prevention of wet age-related macular degeneration.

16. Application of the kit according to claim 9 in the production of an engineered host cell or a population thereof, or a terminally differentiated cell or a precursor cell thereof or a population thereof.

17. Application of CNTF and/or miR-126 expressed by the engineered host cell or the population thereof according to claim 3 in the manufacture of a medicament for the treatment of wet age-related macular degeneration;
preferably, the medicament overexpresses CNTF and/or miR-126;
more preferably, the medicament overexpresses CNTF and miR-126;
preferably, the medicament comprises an agent that overexpresses CNTF and/or miR-126 expression;
preferably, the medicament comprises an agent that overexpresses CNTF and miR-126 expression.

18. Application of CNTF and/or miR-126 expressed by the terminally differentiated cell or the precursor cell thereof or the population thereof according to claim 4 in the manufacture of a medicament for the treatment of wet age-related macular degeneration;
preferably, the medicament overexpresses CNTF and/or miR-126;
more preferably, the medicament overexpresses CNTF and miR-126;
preferably, the medicament comprises an agent that overexpresses CNTF and/or miR-126 expression;
preferably, the medicament comprises an agent that overexpresses CNTF and miR-126 expression.
